(19) United States
(12) Patent Application Publication
(10) Pub. No.: US 2008/0176292 A1
(43) Pub. Date: Jul. 24, 2008

(54) PORTABLE BUOYANCY DRIVEN PCR THERMOCYCLER

(75) Inventors: Victor M. UGAZ, College Station, TX (US); Nitin AGRAWAL, Charlestown, MA (US)

Correspondence Address: CONLEY ROSE, P.C.;David A. Rose, P. O. BOX 3267, HOUSTON, TX, 77253-3267, US

(73) Assignee: TEXAS A&M UNIVERSITY SYSTEM, College Station, TX (US)

(21) Appl. No.: 12/018,570

(22) Filed: Jan. 23, 2008

Related U.S. Application Data
(60) Provisional application No. 60/886,131, filed on Jan. 23, 2007.

(51) Int. Cl. C12P 19/34; C12M 1/00

(52) U.S. Cl. 435/91.2; 435/286.1

(57) ABSTRACT

A closed loop convective flow thermocycler for amplifying DNA sequences via polymerase chain reaction establishes buoyancy driven flow in response to an applied temperature gradient, so that PCR reagents are continuously cycled among temperature zones corresponding to denaturing, annealing and extension temperatures.

### CROSS-REFERENCE TO RELATED APPLICATIONS

This application claims benefit of U.S. provisional application No. 60/886,131 filed Jan. 23, 2007 and entitled Portable Buoyancy Driven PCR Thermocycler which is hereby incorporated herein by reference in its entirety for all purposes.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant Nos. NIH K22-HG02297 and R01-HG003364 awarded by the National Institutes of Health.

### BACKGROUND OF THE INVENTION

1. Technical Field of the Invention

The invention generally relates to apparatus and processes for thermocycling a fluid reactant mixture, and more specifically to such a apparatus and process for DNA amplification via the polymerase chain reaction.

2. Background of the Invention

Polymerase Chain Reaction, hereinafter PCR, is a key technique in molecular biology protocols for the amplification of DNA. In the laboratory, the technique requires a device that cycles the temperature of the reaction suspension through three temperature ranges that allow denaturation, annealing and elongation of the DNA. Typically, these thermocycler devices are large bench top systems that require significant amounts of time (>1 hour). These timescales are not due to reaction kinetics but are primarily a result of the highly inefficient design of conventional thermocycling hardware that remains fundamentally the same today as it was 20 years ago. The hardware for thermocycling typically includes a metal block with electronically variable temperature or mechanically transporting the reagent suspension between blocks or baths at the required temperature range. The metal block design remains the most commonly used, although the time and energy to repeatedly heat and cool the block makes this design extremely inefficient. Furthermore, the complexity of the temperature cycle requires highly specific controls and adjustment which makes PCR thermocyclers expensive pieces of laboratory equipment. In order to fit the temperature cycling and monitoring equipment into one device, PCR thermocyclers may have significant weight. Such limitations necessarily eliminate the capability to rapidly amplify a DNA segment in a field setting due to portability, cost and size requirements.

Micro-device advances and thermocycler research have yielded convection driven thermocyclers. The cavity-based designs typically consist of reactor geometries in which the PCR reagents are enclosed between upper and lower surfaces maintained at annealing and denaturing temperatures, respectively. When the aqueous PCR reagent mixture is heated from below, an unstable “top heavy” arrangement is created which can provide sufficient driving force to establish a continuous circulatory flow in much the same fashion as in an ordinary lava lamp. As the reagent mixture cycles between these temperature ranges, the mixture briefly maintains the elongation temperatures. Further capillary based systems utilize similar schemes for initiating and maintaining the driving convection force. Most notably, these systems' maintenance of two distinct temperature zones that coincide with the annealing and denaturing temperatures of the PCR cycle.

These systems do not actively maintain the temperature necessary for the crucial, and most time consuming elongation portion of the step. Consequently there is a need in the field for a convective device for thermocycling that maintains multiple temperature zones.

### BRIEF SUMMARY

Embodiments of the present apparatus and methods address these and other needs in the art. In accordance with certain embodiments, a PCR thermocycler is provided which comprises a sealable conduit for cycling a reaction fluid through a closed system, from a first temperature zone to a second temperature zone, from a second temperature zone to a third temperature zone, and from a third temperature zone to a first temperature zone. The temperature zones in thermal connection with at least one heating element. At least one heating element maintains the temperature in the zones so that the third temperature zone is higher than the second temperature zone and the second temperature zone is higher than the first temperature zone. The at least one heating element in electrical connection to a temperature controller.

Also provide in accordance with embodiments of the invention is a method of operating the above discussed. The method comprises PCR thermocycler by introducing reagents to the sealed conduit, the conduit having first, second and third sectors that correspond to the first, second and third temperature zones respectively. Heating at least one temperature zone such that the heating causes the fluid reagents to continuously cycle through the sectors of the conduit in the order of first sector to the second sector, from the second sector to the third sector, and from the third sector to the first sector. Thermal transfer between the first temperature zone and the second temperature zone is coupled and controlled by at least one thermal connection. Additionally, thermal transfer between the first temperature zone and the third temperature zone is coupled and controlled by at least one thermal connection. The reaction products are recovered from the conduit.

Thus, embodiments described herein comprise a combination of features and advantages intended to address various shortcomings associated with certain prior devices. The various characteristics described above, as well as other features, will be readily apparent to those skilled in the art upon reading the following detailed description of the preferred embodiments, and by referring to the accompanying drawings.

### BRIEF DESCRIPTION OF THE DRAWINGS

FIG. 1 illustrates a schematic flow diagram of a thermocycling process in accordance with certain embodiments of the invention.

FIG. 2 illustrates a convective flow PCR thermocycler with two active heating elements according to an embodiment of the invention.

FIG. 3 illustrates a schematic of loading reagents into a convective flow PCR thermocycler tube, according to an embodiment of the invention.

FIG. 4 illustrates a convective flow PCR thermocycler with one active heating element, in accordance with an embodiment of the invention.

FIG. 5 illustrates a schematic and a photograph of an embodiment of a convective flow PCR thermocycler.

FIG. 6A illustrates the electrophoresis gel results of PCR amplification of multiple targets in a convective flow PCR thermocycler according to an embodiment of the invention.

FIG. 6B illustrates the electrophoresis gel results of PCR co-amplification of a pair of vastly different length targets in a convective flow PCR thermocycler according to an embodiment of the invention.

FIG. 6C illustrates the electrophoresis gel results of PCR co-amplification of similar length different targets in a convective flow PCR thermocycler according to an embodiment of the invention.

FIG. 6D illustrates the electrophoresis gel results of PCR amplification over different time periods obtained in accordance with an embodiment of the invention.

FIG. 7A illustrates the volumetric flow rate as a function of time versus loop diameter.

FIG. 7B illustrates the master design curve of a triangular convective flow PCR thermocycler.

FIG. 7C illustrates superposition of the master design curves from three different triangular convective flow PCR thermocycler geometries onto a single design curve.

FIG. 8 illustrates a schematic of the concept of a convective flow PCR thermocycler in accordance with certain embodiments of the invention.

### NOTATION AND NOMENCLATURE

Certain terms are used throughout the following descriptions and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection, or through an indirect connection via other devices, materials and connections.

### DETAILED DESCRIPTION OF THE PREFERRED EMBODIMENTS

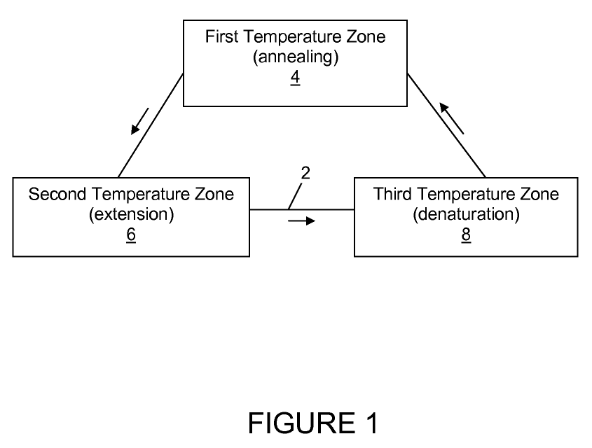

A thermocycler for cycling a reaction fluid through a plurality of temperature zones by convective flow generally comprises a fluid cycle path 2 configured such that the reaction path follows a reaction conduit through a first temperature zone 4 coupled fluidly to a second temperature zone 6; from the second temperature zone 6 coupled fluidly to a third temperature zone 8; and then from the third temperature zone 8 coupled fluidly to the first temperature zone 4. The thermocycler is further comprised of at least one heating element disposed in thermal communication with said temperature zones. The conduit is oriented in a vertical manner with the third temperature zone being located on the upward side of the fluid flow path as schematically illustrated in the block flow diagram of FIG. 1. Without wishing to be limited by convective flow theory, discussed in further detail hereinafter, in operation the reagent fluid is confined within the reaction conduit and as the fluid is heated in the third temperature zone, it becomes buoyant and establishes a convection flow within the conduit that cycles the reaction fluid sequentially through the first, second and third respective temperature zones.

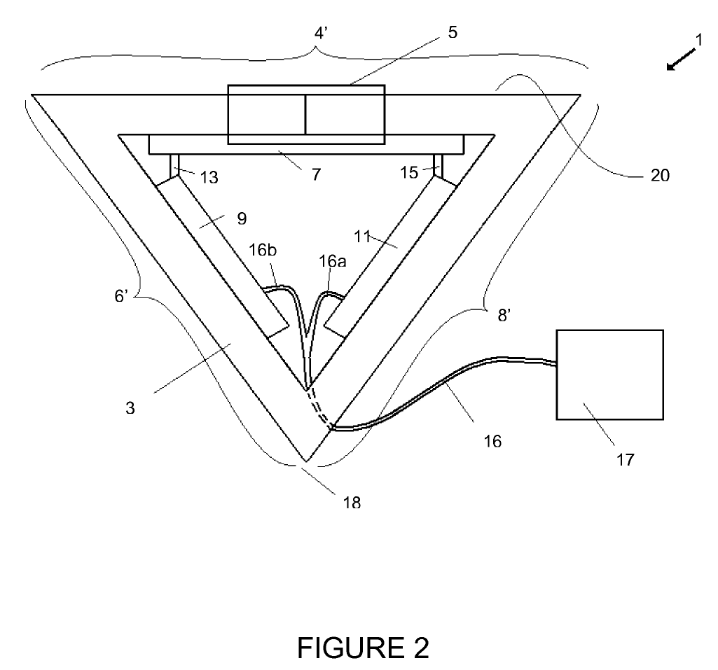

FIG. 2 illustrates an embodiment of a convective flow polymerase chain reaction (PCR) thermocycler 1. The thermocycler 1 is constructed of at least one length of conduit 3 (hereinafter conduit) disposed in a loop. The conduit 3 is disposed in contact a first heating element 7, a second heating element 9, and a third heating element 11 such that the surface of the conduit 3 is in contact with, and thermally coupled to each heating element. Disposed between the heating elements are thermal connections 13, 15 for thermally coupling the heating elements. The thermocycler includes a control 17 for initiating and controlling an electrical current from an electrical power source.

The conduit 3 is a channel, a vessel, tubing or any other structure suitable for circulating a fluid, without limitation. The conduit 3 is a length of tubing. Speaking generally, the heating elements 7, 9, 11 are located within the perimeter of the conduit 3 path. Without wishing to be limited by device design theory, positioning heating elements within the conduit loop makes the device more compact. Alternatively, more than one conduit 3 may be disposed about the heating elements for running a plurality of experiments in parallel. The conduit 3 may be looped to follow any geometric shape, without limitation; in the present embodiment, the tube approximately follows the shape of a triangle.

The conduit 3 is comprised of a thermo-stable polymer. Suitable polymers include fluorinated ethylene propylene, polytetrafluoroethylene, perfluoroalkoxy, or other easily formable, non-reactive polymers as known by one skilled in the art, without limitation. For example the conduit 3 is a linear length of tubing with a length between about 60 mm and about 200 mm, preferably between about 80 mm and about 180 mm and in some exemplary embodiments is between about 80 mm and about 100 mm in length. The conduit 3 also has a diameter of about 200 μm and about 600 μm, preferably between about 300 μm and about 550 μm, and in some exemplary embodiments is between about 320 μm and about 510 μm. The volume of liquid reagents the conduit 3 may hold is preferably between about 5 μl and about 100 μl, and in exemplary embodiments between about 10 μl and about 25 μl. It can be envisioned that conduit 3 may be sized to accommodate different volumes of liquid, based on the time required for amplification of a particular DNA fragment, as determined using PCR techniques. Additionally, the conduit 3 can be optically transparent so as to facilitate optical analysis and product detection during the reaction. Said optical analysis may be any protocol known to one skilled in the art, such as confocal microscopy, fluorescence detection, or laser probing.

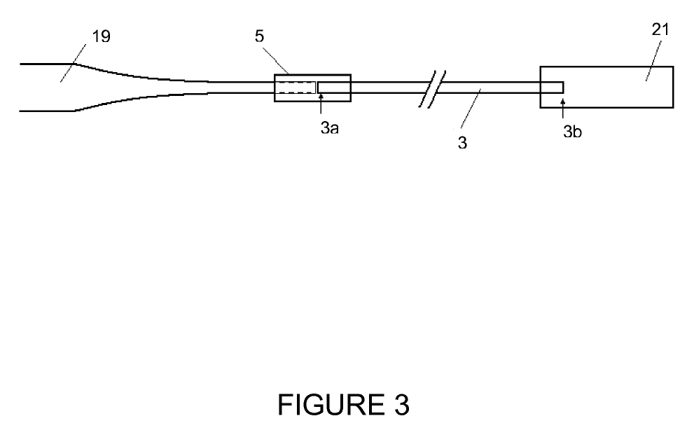

Referring now to FIG. 3, illustrating an assembly for loading the conduit 3 of FIG. 1 with a reagent fluid. The conduit 3 has a first end 3a associated with a sleeve 5, and a second end, or free end 3b. The sleeve 5, disposed at the associated end 3a of the conduit 3 is a means to reversibly seal the conduit 3 into a closed system. In the illustrated assembly the sleeve 5 facilitates loading of reagents into the conduit 3. The sleeve 5 aligns the tube 3 with a vessel 19 for the introduction of reagents into the tube 3. In certain embodiments, the vessel 19 is a pipet tip. A vessel 19 is inserted into the sleeve to load the reagents into the tube. The free end 3b of the tube 3 is disposed in a volume of the reagents held by a fluid container 21. As illustrated a vacuum applied to the vessel 19 in the sleeve 5 draws reagents from the fluid container 21 into the conduit 3. The fluid container 21 can be any suitable container, such as without limitation, a test tube, a beaker, a centrifuge tube, a BD Falcon™ tube, or a vial. When the conduit 3 is filled, the free end 3b of the conduit is then looped to form a closed system by inserting it into the sleeve 5 until it meets with the associated end 3a. Over loading the conduit 3 to the extent that excess fluid is squeezed out when the ends 3a, 3b are joined reduces or eliminates air pockets within the conduit 3.

In some configurations the sleeve is disposed anywhere along the closed length of the conduit 2. In alternate configurations, it can be envisioned a needle, or other delivery device without limitation punctures the sleeve 5 and injects the reagents into the system. In this design, the sleeve 5 acts as a self sealing point on the conduit 3 for injection of the reagents by needle. Additionally, the sleeve 5 may include additional structures such as an access membrane, valve, flip-top, port or similar construction as known to one skilled in the art, for a re-sealable introduction, or withdrawal, point for a fluid. The sleeve 5 is absent from the conduit 3 such that the system is closed, sealed or looped continuous system in alternative constructions.

As illustrated in FIG. 2, the conduit 3 is disposed in a loop in thermal contact with the first heating element 7, the second heating element 9, and the third heating element 11 in sequence. As stated previously, the fluid conduit 3 circulates the reaction fluid through a first temperature zone coupled fluidly 4 to a second temperature zone 6; the second temperature zone 6 coupled fluidly to a third temperature zone 8; the third temperature zone coupled fluidly to the first temperature zone. The first temperature zone 4 comprises a first heating element 7; the second temperature zone 6 comprises a second heating element 9 and the third temperature zone comprises a third heating element 11. As shown in this conformation, the conduit 3 forms a triangle, oriented vertically on one corner 18, with the opposing side generally oriented horizontally 20 and elevated with respect to the position of corner 18.

The heating elements 7, 9, 11 are a device that increase temperature of adjacent space, materials or structures respectively. In certain configurations, the heating elements 7, 9, 11 are positioned adjacent to, partially surrounding, or entirely enclosing the intended thermal recipient, the conduit 3. Generally, the heating elements comprise materials such as metal blocks, alloy blocks, wire filaments, coils or alternate constructions may be a heating element without limitation. A heating element may be a passive heating element or an active heating element. A passive heating element may be any thermally conductive material suitable for transferring heat to the conduit 3 from another source. A passive heating element may include a thermal connection to an active heating element to maintain a preferred temperature range. An active heating element maintains connection to an electrical power source controller 17 that regulates electrical current to the active heating element via lead 16. The electrical power may be provided by any known source, without limitation. In certain instances, the electrical power source is a battery or other electric power storage device. A power storage device eliminates the need for the thermocycler 1 to be connected to an outlet, and can allow the thermocycler 1 to be portable. The controller 17 determines the amount of electricity that reaches the active heating element or elements, and the temperature thereof. Examples of active heating elements include, without limitation, a heat pump, electrical resistance heater or other such devices that convert electrical energy to heat. In preferred embodiments, a Peltier heater is used as an active heating element, for the second heating element 9 and the third heating element 11.

In FIG. 2, heating element 7, of the first temperature zone 4, is a passive heating element. Further, the second temperature zone 6 comprises heating element 9 and the third temperature zone heating 8 comprises heating element 11, as illustrated, are active heating elements coupled to lead 16 by leads 16a and 16b respectively. For thermal communication from active heating elements 9, 11, the first heating element 7 has a thermal connection 13 to the second heating element 9 and the first heating element 7 has a thermal connection 15 to the third heating element.

The thermal connections 13, 15 are constructed out of any material suitable for conducting heat. Examples include metals, alloys, ceramics, polymers or other materials without limitation. The thermal connection may be a structure that is capable of being changed, moved, or removed completely from the thermocycler 1. Adjusting the position of the thermal connection between heating elements allows control of the heat transfer between active and passive elements. A screw, a pin, a washer, a post or a wire are all suitable examples for thermal connection between the first heating element 7 and the second heating element 9 or third heating element 11. Alternatively in some applications, a suitable air space between an active element and a passive element conducts thermal energy sufficiently to maintain the temperature of the passive heating element.

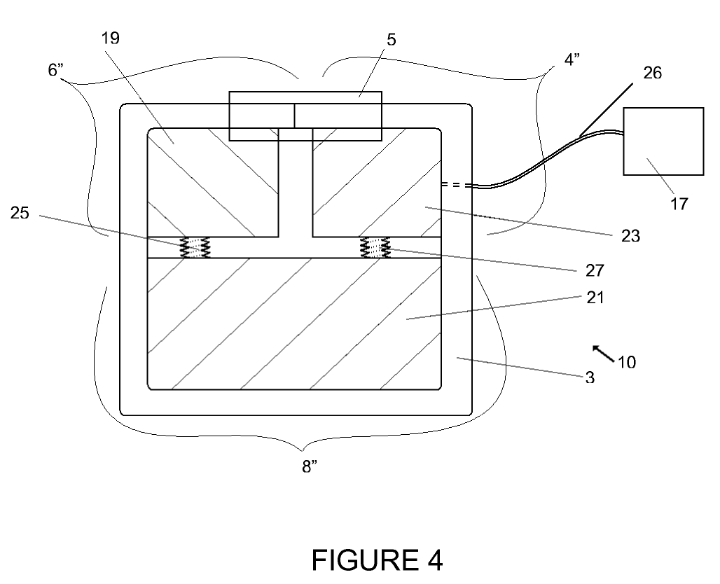

Referring now to FIG. 4, an alternative configuration of the thermocycler is shown, in which the conduit 3 forms a generally quadrilateral shape. Analogous parts of the construction to that which has been previously described include a conduit 3 with associated sleeve 5. The conduit 3 is disposed around a first heating element 19, a second heating element 21, and a third heating element 23 sequentially such that the conduit 3 is in thermal communication with each said heating element. Disposed between the heating elements are thermal connections 25, 27 for thermally coupling the heating elements. In the illustrated configuration the thermal connections 25, 27 are metallic screws. Similar to prior discussed configurations, active heating elements have an electrical control 17 for initiating and controlling an electrical current from an electrical power source.

In the thermocycler 10 configuration illustrated in FIG. 4, the first heating element 19 is a passive heating element, the second heating element 21 is a passive heating element, and the third heating element 23 is an active heating element. The passive heating elements 19, 21 are thermally coupled to the third heating element 23 in sequence. As illustrated a resistive block heater is used as the third heating element 23. The third heating element 23 may be a heat pump, electrical resistance heater or other such device that converts electrical energy to heat. Element 23 is coupled to controller 17 via lead 26. Without wishing to be limited by thermal conduction theory, the heat is transferred to the second heating element 21 by a thermal connection 27. Further, the second heating 21 element transfers heat to the first heating element 19 by thermal connection 25.

Each subsequent thermal connection has a specific thermal conductivity that allows the passive heating elements to maintain a range of temperatures favorable for the reaction in the adjacent tube 3. The thermal connections 25, 27 are thermally conductive screws that can be adjusted to determine the amount of contact and consequently heat transfer between each heating element. Further, adjusting the thermal connections 25, 27 allows for the independent temperature control in each heating element. The use of a mechanical thermal connection, such as a screws and blocks where they are chosen to have the desired thermal conductivity, imparts the capacity to regulate temperatures in each zone in an independent manner. Mechanical connections require no further energy to monitor or maintain the temperature in each zone, there for making the device more efficient. Additionally, mechanical thermal connections increase the durability of the thermocycler in field conditions, such that real time data can be obtained outside of a laboratory.

In these configurations, shown in FIGS. 2 and 4, the first heating element 7, 19 maintains a temperature of between about 50° C. and about 65° C. in the first temperature zone 4, 4′. The second heating element 9, 21 maintains a temperature between about 70° C. and about 75° C. in the second temperature zone 6, 6′. The third heating element 11, 23 maintains a temperature between about 92° C. and about 100° C. in the third temperature zone 8, 8′. It can be envisioned that for further thermocycling applications the temperature ranges in each zone may be adjusted for that application; additional temperature zones may be created by differential control of thermal connections, electrical power input and the heating elements.

Without wishing to be limited by theory regarding polymerase chain reaction the thermocycling of the reagents controls the process of amplification of a particular target DNA sequence by utilizing the complementary single DNA strands. These DNA strands of the sequence function as templates for the enzyme to synthesize a new strand. The reagents for the process include individual nucleic acid bases, long complimentary single stranded DNA template sequences to be amplified, short single stranded DNA primers and a thermo-stable polymerizing enzyme. Sequentially, the process occurs in the following order: single stranded DNA templates in solution are annealed with single stranded primers at the temperature range controlled by the first heating element 7, 19 in zone 6′,6″. The primers are a short sequence of complimentary base pairs that aid in positioning the enzyme at the start of the template single strand of DNA. Typically the primers are a short strand of DNA. The enzyme is preferentially active within the range of temperatures maintained by the second heating element 9. At this temperature, the enzyme is capable of catalyzing the extension of a DNA strand at about 50 base pairs to about 120 base pairs a per second. The reaction products of the enzymes action is a double stranded, or complementary, sequence fragment of DNA. The newly synthesized double stranded DNA is denatured or separated into the complementary pairs of single stranded DNA templates within the temperature range controlled by the third heating element 11, 23 of zone 4′, 4″. The process doubles the quantity of complementary pairs of single stranded DNA templates with every cycle through the three temperature ranges. The reaction products become the reagents, specifically the singe stranded DNA template, for the subsequent rounds of the reaction. After a sufficient cycling time has passed to amplify the target DNA, as determined using the technique of PCR technique, the amplified target DNA is recovered from the conduit in the opposite manner of introduction.

In embodiments the reagents are cycled through the tube such that the reagents encounter each of the temperature zones via convectional flow. Without wishing to be limited by any particular theory to explain the mechanism of the process, the higher temperature, less dense zone of reagent mixture in the tube 3, rises due to buoyancy allowing reagents from a lower temperature, denser zone to occupy the space previously occupied by the higher temperature liquid. Description of the principles of buoyancy driven convective flow reactions are covered in U.S. Patent Application Publication No. 2005/0074782 A1, and further details of these principles are described by M. Krishnan, V. M. Ugaz and M. A. Burns (“PCR in a Rayleigh-Benard convection cell,” Science 298: 793, Oct. 25, 2002), hereby incorporated herein by reference in entirety for all purposes. In summary, steady circulatory flow is generated such that it involves the entire volume of the liquid, and maintains a flow velocity that allows enough time in each temperature zone to allow reaction completion with each cycle.

In other embodiments, a thermocycler for simultaneously processing multiple target DNAs comprises a multiple respective conduits in contact with the heating elements, positioned adjacent to the conduit 3. In still other embodiments, a conduit similar to the conduit 3 has alternate compositions or constructions such that it acts as an analytical tool for another molecular biology or biochemistry protocol that may precede or follow amplification via PCR as described herein. In certain instances, dried or lyphophilized reagents are stored in the conduit 3 until need for analysis, whereupon the desired fluid is introduced into the conduit to solubilize the reagents.

While the preferred embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims.

To further illustrate various illustrative embodiments of the present invention, the following examples are provided.

### EXAMPLE

An ideal PCR system should be capable of amplifying a wide range of targets in both single and multiplex formats. Unfortunately, the timescales and complexities involved in many existing technologies impose significant limitations on achievable throughput. A simplified convectively driven thermocycler is capable of performing single and multiplex PCR for amplicons ranging from 191 bp to 1.3 kb within 10 to 50 minutes using 10 to 25 μl reaction volumes. By positioning two Peltier heaters along the perimeter of a flow loop reactor constructed using ordinary plastic tubing, a buoyancy-driven flow is established that continuously circulates reagents through temperature zones associated with the PCR process. Unlike conventional benchtop thermocyclers, this arrangement allows reactions to be performed without the need for dynamic temperature control of inactive hardware components while maintaining comparable product yields and requiring no modifications to standard PCR protocols. A general correlation that can be applied to design reactor geometries satisfying virtually any combination of reagent volume and cycling time. This system offers an attractive combination of cost and performance and is readily adaptable for portable battery powered operation.

A simple microfluidic thermocycler is designed to perform rapid polymerase chain reaction (PCR) in single and multiplex formats. This system is also capable of replicating long amplicons (>1 kb) making it relevant for whole-genome analysis. The device operates by harnessing a static temperature field to generate unidirectional buoyancy-driven convective flow in a closed loop reactor that continuously circulates reagents through the PCR temperature zones. Convective PCR offers a compelling emerging approach to address many of the limitations of conventional dynamically controlled thermocyclers (e.g., slow cycling times, excessive power consumption). Previous studies, however, have been primarily directed at the proof-of-concept level, often employing cumbersome designs with relatively long cycling times and/or large reactor volumes that are not practical for routine use. Furthermore, successful amplification has only been-demonstrated over a limited range of template and target sizes and has not included multiplex capability.

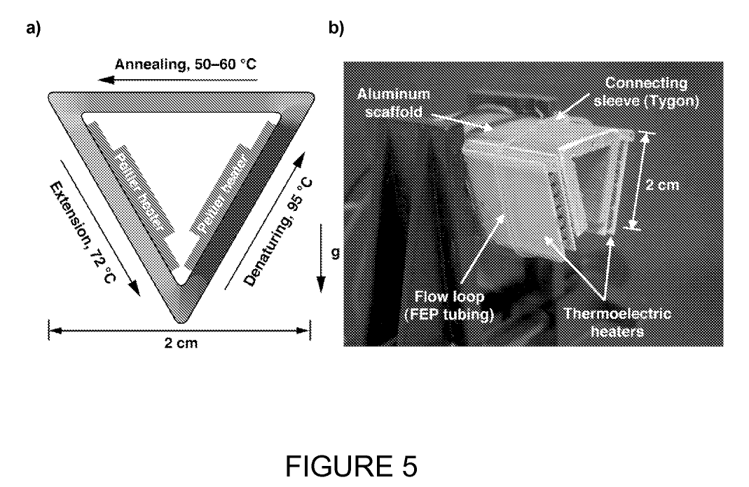

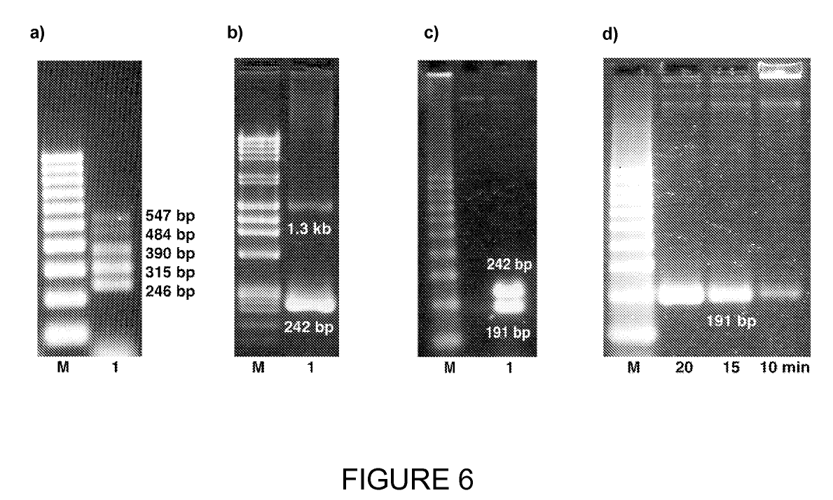

A greatly simplified closed loop convective flow thermocycler is constructed by positioning two Peltier heaters along the perimeter of a Teflon tubing reactor in a triangular arrangement as shown in FIG. 5A. The heaters are independently controlled to maintain denaturation (95° C.) and extension (72° C.) temperatures across the two inclined legs, while the horizontal leg passively attains the annealing (60° C.) temperature. FIG. 5B shows a prototype construction of the convective flow thermocycler. As shown, there is potential to run multiple closed loops around the Peltier heaters and aluminum support. Thermocycling is automatically performed by the unidirectional convective flow generated in response to the imposed thermal gradient. This system is capable of performing PCR amplification in as little as 10 min as in FIG. 6A, and robustness is demonstrated by successfully amplifying a variety of template—target combinations in both single and multiplex formats using 10-25 μL reaction volumes. Product yields are comparable to those achievable in conventional benchtop thermocyclers, and no modifications to standard reaction protocols are necessary. Reactor geometries can be strategically designed to satisfy virtually any desired combination of reagent volume and cycling time.

The versatility of this convective flow thermocycling system is demonstrable by using it to amplify a multiplex mixture of targets associated with 5 different respiratory viruses in 50 min using a 16 μL (400 μm diameter) flow loop reactor (FIG. 6A). Simultaneous co-amplifications in mixtures containing primers and template for a 1.3 kb target from a λ-DNA template and a 242 bp human L32 gene target (FIG. 6B), as well as 191 bp influenza-A virus and 242 bp human L32 gene targets (FIG. 6C) in 50 min using a 16 μL loop are possible. The ability to handle a broad range of target lengths is further demonstrated by using a 10 μL (320 pm diameter) loop to amplify a 1.3 kb target from a λ-DNA template and a 474 bp human β-Actin target in 50 min. The fluoropolymer tubing used to construct the flow loops is chemically inert and thus exhibits a low susceptibility to non-specific binding interactions, eliminating the need for surface pre-treatment processes required in many miniaturized PCR systems.

The ability to perform rapid amplification is critically important in many applications (e.g., medical diagnostics, pathogen detection assays). By characterizing amplification efficiency in 10, 16, and 25 μL (320,400 and 510 pm diameter, respectively) closed loop reactors with corresponding cycling times of 102, 69, and 42 s, respectively (flow velocities in the loops scale with tubing diameter) is shown. Using the 25 μL loop, amplification of a 191 bp target was detectable by agarose gel analysis after as little as 10 min of reaction time (FIG. 6D). Smaller volume flow loops (i.e., constructed from smaller diameter tubing) delivered longer cycling times and produced detectable products in 20-30 min. This level of performance is significant because cycling times approaching those demonstrated in the most rapid thermocycling approaching those demonstrated in the most rapid thermocycling systems to date can be achieved in a device format incorporating an unprecedented level of mechanical and electronic simplicity.

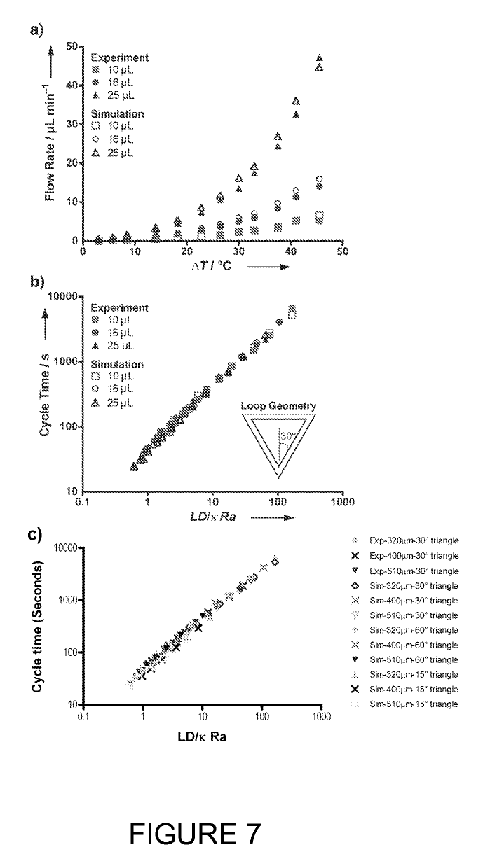

A simplified analytical model is presented that highlights the underlying physics and establishes general design criteria for convective loop thermocyclers. The major physical parameters influencing convective loop flows become evident by first noting that the magnitude of the destabilizing buoyant forces relative to the dimensionless Rayleigh number Rα=(g·cos θ)β(ΔT)D³/vκ. Here, g·cos θ is the component of gravitational acceleration acting along the flow direction (i.e., θ is the angle a segment of the flow loop makes with respect to the vertical), ΔT is the temperature difference imposed between the two heaters, D is the tube diameter, L is the loop length, and β, κ, and [symbol] are fluid properties (thermal expansion coefficient, thermal diffusivity, and kinematic viscosity, respectively). Consequently, the magnitude of Rα (and hence the flow velocity) increases with increasing ΔT and with increasing tube diameter, consistent with data obtained by observing the motion fluorescent microsphere tracers inside the flow loops (FIG. 7A). In addition, the onset of fluid motion occurs upon application of a very small temperature difference (ΔT˜1° C.). Under PCR conditions, average flow velocities of approximately 0.9, 1.3, and 2.1 mm/sec were obtained in 10, 16 and 25 μL loops, respectively.

Over the range of flow conditions of interest for PCR, analysis of the momentum and energy balance relationships for the case of a loop geometry symmetric about the vertical centerline (e.g., the triangular configuration in FIG. 1a) suggests that Rα should scale with uD/κ (u is the average velocity; see Supporting Information). This scaling is evident when cycling times determined both from flow visualization experiments and from computational simulations are plotted versus the parameter LD/κRα, where data for various reactor volumes collapse onto a single curve (FIG. 7B). The scalings obtained for velocity and cycle time should be generally applicable to loop configurations that are symmetric about a vertical centerline. This is supported by the results of computational flow simulations for two additional symmetric triangular loop geometries (θ=15 and 60°) where the total loop length L kept fixed at 9 cm as in the θ=30° triangular configuration used to perform PCR. It can be seen that data for all three geometries collapse onto a master curve consistent with the expected scaling (FIG. 7C).

This relationship permits strategic design of reactors satisfying virtually any combination of volume and cycling time. For example, the cycle time achievable at a fixed loop length can be estimated using FIG. 7B by first computing LD/κRα at a desired D and ΔT. Conversely, the loop geometry and reactor volume required by first specifying the desired cycle time and using the master curve to determine the magnitude of LD/κRα.

In addition to adjusting the global loop geometry (i.e, D, L) it is also possible to incorporate local modifications in order to deliver different residence times under denaturing, annealing, or extension conditions. The modifications can include larger or smaller tube diameters at different locations along the flow path, or a zigzag flow path within one or more of the temperature zones. Finally, although the laminar parabolic flow profile implies a variation in velocity over the tube cross section (e.g., slower velocities near the wall and faster speeds at the centerline), these effects are counteracted by lateral molecular diffusion of reagents across streamlines and perturbations to fluid trajectory at the corners of the flow path.

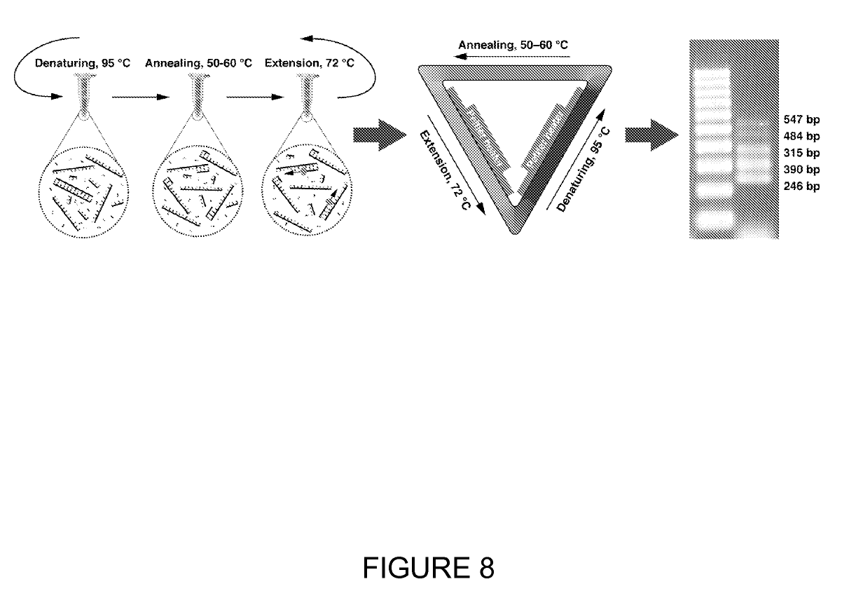

The convective flow thermocycling systems have advanced beyond the proof-of concept phase of development, and are now capable of seriously competing with existing mainstream instrumentation. The attractive combination of performance and simplicity offered by convective thermocycler design, as illustrated in the schematic in FIG. 8, addresses many of the shortcomings of current technology, ultimately making it feasible to greatly expand the use of PCR-based assays. In addition to extremely low hardware costs, the reduced level of power consumption and elimination of the need for dynamic temperature control also make this system ideal for use in portable battery operated applications.

The discussion of a reference in the Description of the Related Art is not an admission that it is prior art to the present invention, especially any reference that may have a publication date after the priority date of this application. The disclosures of all patents, patent applications, and publications cited herein are hereby incorporated herein by reference in their entirety, to the extent that they provide exemplary, procedural, or other details supplementary to those set forth herein.

### REFERENCES

1. U.S. Pat. No. 5,919,622 (Macho et al.)

2. U.S. Pat. No. 6,586,233 (Benett et al.)

3. U.S. Pat. App. Pub. 2005/0074782 (Krishnan et al.)

4. Roper M G, C J Easley and J P Landers, “Advanced in polymerase chain reaction on microfluidic chips,” Anal Chem 77:3887-3894 (2005).

5. Krishnan, M, V M Ugaz and M A Burns, “PCR in a Rayleigh-Benard convection cell,” Science 298: 793 (Oct. 25, 2002).

6. Krishnan M, N Agrawal, M A Burns and V M Ugaz, “Reactions and fluidics in miniaturized natural convection systems,” Anal Chem 76:6254-6265 (2004).

7. Ugaz V M and M Krishnan, Technical Brief: “Novel convective flow based approaches for high-throughput PCR thermocycling,” JALA 9:318-23 (2004).

8. Hennig M and D Braun, “Convective polymerase chain reaction around micro immersion heater,” App Phys Letts 87:18901 (2005).

9. Braun, D, N L Goddard and A Libchaber, “Exponential DNA replication by laminar convection,” Phys Rev Lett 91:158103 (2003).

10. Wheeler, E K, W Benett, P Stratton, J Richards, K D Ness, J Ortega, L G Li, T H Weisgraber, K Goodson and F Milanovich, “A Chen, A Christian, K D “Convectively driven polymerase chain reaction thermal cycler,” Anal Chem 76:4011-4016 (2004).

11. Chen, Z, S Qian, W R Abrams, D Malamud and H H Bau, “Thermosiphon-based PCR reactor: experiment and modeling,” Anal Chem 76:3707-3715 (2004).

12. Agrawal, N. and Ugaz, V. M. “A Battery Powered Compact Thermocycler for Rapid PCR.” Poster MP02. Lab Automation 2006. Palm Springs, Calif., Jan. 23, 2006 (1 pg).

13. Agrawal, N. and Ugaz, V. M. “A Battery Operated Portable Thermocycler for Rapid PCR.” Poster Session C. 23^{rd} Annual Houston Conference on Biomedical Engineering Research. Houston, Tex., Feb. 9, 2006 (1 pg).

14. Agrawal, N and V M Ugaz, “A buoyancy-driven compact thermocycler for rapid PCR,” JALA 11:217-21 August 2006.

15. Agrawal, N and V M Ugaz, “A Pocket-sized Convective PCR Thermocycler.” Angew. Chemie Int. Ed. 46: 4316-4319 (2007).

16. Spitzak, K D and Ugaz, V M, “Polymerase chain reaction in miniaturized systems: big progress in little devices,” Methods in Molecular Biology 321:97-129 (2006).

What is claimed is:

1 A thermocycling device comprising:
in a series fluid flow arrangement, a first temperature zone, a second temperature zone, and a third temperature zone;
at least one fluid conduit configured for circulating fluid sequentially through said first, second and third temperature zones, wherein at least said third temperature zone comprises a heating element;
a controller coupled to said at least one heating element;
wherein said at least one heating element is in thermal communication with a respective portion of said conduit; and
wherein said device is configured to maintain the temperatures of said respective portions in said zones in the order of first temperature zone, is less than the second temperature zone, is less than the third temperature zone, is greater than the first temperature zone.

2. The device of claim 1, wherein each said zone comprises a heating element, in thermal communication with respective portions of conduit.

3. The device of claim 1, wherein at least one said heating element is an active heating element in thermal communication with said third temperature zone.

4. The device of claim 2 wherein the at least one heating element comprises a thermoelectric heater.

5. The device of claim 3 wherein the said first temperature zone comprises at least one passive heating element in thermal communication with the said active heating element.

6. The device of claim 1 wherein said second temperature zone comprises at least one said passive heating element in thermal communication with said third temperature zone.

7. The device of claim 1 wherein the fluid conduit is configured as a continuous fluid conduit with closure member configured for sealing the internal environment from the external environment.

8. The device of claim 1 further comprising an electric controller coupled to said at least one heating element.

9. The device of claim 1 further comprising at least one thermal transfer member configured for controlling thermal communication between at least two said temperature zones.

10. The device of claim 1 wherein said device is portable and includes an electrical power source.

11. A method for thermocycling a fluid, comprising:
providing the device of claim 1;
containing fluid reagents in at least one conduit, wherein said conduit comprises first, second, and third sectors, said sectors in thermal communication with respective first, second and third temperature zones;
heating at least one said temperature zone with at least one heating device, whereby said heating causes the fluid reagents to continuously cycle in said conduit from said first conduit sector to said second conduit sector, from said second conduit sector to said third conduit sector, said third conduit sector to said first conduit sector;
controlling thermal transfer between the first temperature zone and the second temperature zone, whereby the at least one heating element of the first temperature zone is coupled to at least one heating element of the second temperature zone by a first thermal connector, whereby thermal communication from said first temperature zone heating element to said second temperature zone heating element is controlled by said first thermal connector;
controlling thermal transfer between the first temperature zone and the third temperature zone, whereby the at least one heating element of the first temperature zone is coupled to at least one heating element of the third temperature zone by a second thermal connector, whereby thermal communication from said first temperature zone heating element to said third temperature zone heating element is controlled by said second thermal connector; and
recovering the reaction products.

12. The method of claim 11, wherein introducing fluid reagents into a conduit comprises introducing polymerase chain reaction reagents, whereby said reagents include a double stranded nucleic acid, a single stranded nucleic acid, and an enzyme.

13. The method of claim 11, wherein heating said third temperature zone to a temperature effective to denature any double stranded nucleic acid in said first conduit sector comprises maintaining a temperature in the range of about 92° C. to about 100° C. in said first conduit sector.

14. The method of claim 11 wherein heating said second temperature zone to a temperature effective to allow annealing of said primers and target nucleic acid in said second conduit sector comprises maintaining a temperature in the range of about 50° C. to about 60° C. in said second conduit sector.

15. The method of claim 11 wherein heating said first temperature zone to a temperature effective to allow extension of any annealed target nucleic acid by the enzyme in said third conduit sector comprises maintaining a temperature in the range of about 70° C. to about 75° C. in said third conduit sector.

16. The method of claim 11 wherein the thermal communication from said first temperature zone to said third temperature zone is controlled by mechanically altering at least one said second thermal connector between the first heating element and the third heating element.

17. A polymerase chain reaction process comprising:
introducing a reaction fluid into the thermocycling device of claim 1, wherein said conduit comprises first, second and third portions, said portions being in thermal communication, respectively with a first temperature zone, second temperature zone and third temperature zone, wherein said reactant fluid comprises a target DNA for amplification, oligonucleotide primers, polymerase, nucleotide triphosphates, and a buffered aqueous solution;
heating said third temperature zone to a temperature effective to denature any double stranded nucleic acid in said first channel portion;
heating said second temperature zone to a temperature effective to allow annealing of said primers and target nucleic acid in said second channel portion;
heating said first temperature zone to a temperature effective to allow extension of any annealed target nucleic acid in said third channel portion,
whereby said reactant fluid in said channel is caused to continuously circulate said fluid from said first portion, to said second portion, from said second portion to said third channel portion, and from said third portion, to said first portion; and
recovering the resulting amplified target nucleotide.

18. The method of claim 17 wherein heating said third temperature zone to a temperature effective to denature any double stranded nucleic acid in said first channel portion comprises maintaining a temperature in the range of about 92° C. to about 100° C. in said third channel portion.

19. The method of claim 17 wherein heating said second temperature zone to a temperature effective to allow annealing of said primers and target nucleic acid in said second channel portion comprises maintaining a temperature in the range of about 50° C. to about 60° C. in said second channel portion.

20. The method of claim 17 wherein heating said first temperature zone to a temperature effective to allow extension of any annealed target nucleic acid in said third channel portion comprises maintaining a temperature in the range of about 70° C. to about 75° C. in said first channel portion.

21. A kit for performing PCR, comprising:
a sealable conduit having opposing ends and configured for receiving a fluid; and
a thermocycling device configured for receiving said conduit when said opposing ends are sealably coupled,
wherein said thermocycling device comprises:
in series fluid flow arrangement, a first temperature zone, a second temperature zone, and a third temperature zone configured for circulating a fluid reagent mixture sequentially through said first, second and third temperature zones, wherein at least said third temperature zone comprises a heating element, and
a controller coupled to said heating element.
